(19) United States
(12) Patent Application Publication
(10) Pub. No.: US 2007/0243295 A1
(43) Pub. Date: Oct. 18, 2007

(54) Spore Inactivation Process

(75) Inventors: Andrew Scully, New South Wales (AU); Katherine Zerdin, New South Wales (AU); Michael Rooney, New South Wales (AU); Ailsa Hocking, New South Wales (AU); Cynthia Stewart, Summit- Argo, IL (US)

Correspondence Address: MINTZ, LEVIN, COHN, FERRIS, GLOVSKY;AND POPEO, P.C., ONE FINANCIAL CENTER, BOSTON, MA, 02111, US

(73) Assignee: COMMONWEALTH SCIENTIFIC AND INDUSTRIAL RESEARCH OR, Australian Capital Territory (AU)

(21) Appl. No.: 10/578,215

(22) PCT Filed: Nov. 3, 2004
(86) PCT No.: PCT/AU04/01521

§ 371 Date: Jun. 11, 2007

(30) Foreign Application Priority Data
Nov. 3, 2003 (AU) 2003906074

(51) Int. Cl. A23L 3/015; A23L 3/3436

(52) U.S. Cl. 426/392; 426/416

(57) ABSTRACT

The present invention relates to processes for inactivating microbial spores in packaged materials including foods pharmaceuticals and cosmetics, by a combination of ultra high pressure processing and oxygen absorption.

### FIELD OF THE INVENTION

The present invention relates to high pressure processing of foods and other products susceptible to microbiological contamination.

### BACKGROUND OF THE INVENTION

High pressure processing (herein “HPP”; also known as “high pressure treatment” or “ultra-high pressure treatment” or “ultra-high pressure sterilization”) is a process that may involve the application of pressures in the range of 100-1,000 MPa (14,500-145,000 psi) to eliminate vegetative cells of bacteria, mould and the like from products where these cells exist.

HPP finds particular application in the food industry because of a capacity to eliminate vegetative cells with minimal heat treatment, resulting in the almost complete retention of nutritional and sensory characteristics of fresh food without sacrificing shelf-life. Other advantages of HPP over traditional thermal processing include reduced process times, minimal heat damage problems, retention of freshness, flavour, texture, and colour and no vitamin C loss.

HPP may find application in other industries where the elimination of vegetative cells is required, for example, pharmaceutical and cosmetic industries.

Some groups have sought to modify HPP so as to minimise the effect of the process on the nutritional and sensory characteristics of food. Specifically, although not as deleterious to these characteristics as thermal treatments such as canning, the nutritional and sensory characteristics of food may be affected by HPP, where to obtain an appropriate shelf life for a food, a pressure treatment is required that is relatively severe in terms of the size of the pressures that are applied, or the length of time over which pressure is applied.

Japanese publication no. 5-7479, A (Toppan Printing Co., Ltd) is directed to a modification of HPP in which pressure treatments are minimised so as to retain the nutritional and sensory characteristics of food without sacrificing the lethal effect of HPP on vegetative cells of bacteria According to 5-7479, a key feature of the process is the elimination of oxygen inside a container that contains a food product and the elimination of oxygen dissolved in a food product. According to 5-7479, the elimination of oxygen is essential because oxygen reduces the bactericidal effect of HPP. According to 5-7479, vacuum treatment and inert gas flushing are the preferred processes for elimination of oxygen. In contrast, according to 5-7479, oxygen scavengers are not suitable for the elimination of oxygen inside a container that contains a food product or for eliminating oxygen dissolved in a food product.

One limitation of HPP is that to date it has been difficult to achieve commercial sterility of a food product with this process. This is because microbiological spores, such as bacterial and mould spores, tend to be resistant to pressure treatment.

U.S. Pat. Nos. 6,207,215 and 6,086,936 (Kal Kan Foods Inc.) are directed to a modification of HPP in which foods are pressure treated at an elevated temperature to achieve an adiabatic temperature increase.

A number of other modifications of HPP have been proposed including combining pressure treatment with an alternating current, ultrasonic frequency, additives such as enzymes, and pressure cycling.

Some of these processes tend to affect the nutritional and/or sensory characteristics of food.

### SUMMARY OF THE INVENTION

In one aspect, the present invention provides a method for inactivating a microbiological spore including the steps of subjecting a microbiological spore to an ultra high pressure treatment (also known as “high pressure processing”, “high pressure treatment” or “ultra high pressure sterilization”) and absorbing oxygen from an environment about the spore to at least limit the consumption of oxygen by the spore.

In another aspect, the present invention provides a method for producing a packaged food product including the steps of adding a food product to a package and subjecting the food product to an ultra-high pressure treatment wherein the food product is in an oxygen-scavenging environment either before or after the ultra-high pressure treatment, said ultra-high pressure treatment and said oxygen scavenging environment being selected for inactivation of a selected microbiological spore in the food product.

In another aspect, the present invention provides, in a process for manufacture of a food product, the steps of subjecting a food to an ultra-high pressure treatment and absorbing oxygen from an environment about the food to provide conditions for limiting the consumption of oxygen by a microbiological spore in the environment.

In another aspect, the invention provides a food product manufactured by the above described process.

In another aspect, the present invention provides a use of an oxygen scavenger for inactivating a microbiological spore in an ultra-high pressure treatment of a food product.

In another aspect, the present invention provides an ultra-high pressure treatment adapted for inactivating a microbiological spore in a food product, the treatment including the step of absorbing oxygen from an environment about the food product to provide conditions for limiting the consumption of oxygen by a microbiological spore in the environment.

In another aspect, the invention provides a method for achieving commercial sterility of a product (i.e. preventing microbiological growth in or on the product) including the steps of subjecting a product to an ultra-high pressure treatment and absorbing oxygen from an environment about the product to provide conditions for limiting the consumption of oxygen by a microbiological spore in the environment.

In another aspect, the invention provides a product produced by the above described method.

### BRIEF DESCRIPTION OF THE FIGURES

FIG. 1. Effect of oxygen scavenging on survival of Baccilus subtilis spores in nutrient broth contained in pouches and stored at 30° C., with (above) and without (below) high-pressure processing.

FIG. 2. Effect of oxygen scavenging on survival of Neosartorya fischeri spores in nutrient broth contained in pouches and stored at 30° C., with (above) and without (below) high-pressure processing.

FIG. 3. Effect of initiating oxygen scavenging before (“pre HPP”) or after (“post HPP”) high-pressure processing of B. subtilis spores in nutrient broth contained in pouches and stored at 30° C.

FIG. 4. Effect of initiating oxygen scavenging before (“pre HPP”) or after (“post HPP”) high-pressure processing of N. fischeri spores in nutrient broth contained in pouches and stored at 30° C.

### DETAILED DESCRIPTION OF THE EMBODIMENTS

In certain embodiments, the inventors have sought to modify high pressure processing (herein “HPP”; also known as “high pressure treatment” or “ultra-high pressure treatment” or “ultra-high pressure sterilization”) so that these processes can be used to inactivate, or in other words, to at least limit and preferably, to prevent the germination of aerobic microbiological spores of bacteria, mould and the like, or otherwise, so that these processes can be used to limit or prevent a germinated or partially germinated spore from becoming a cell, especially a cell that is capable of vegetation. As described herein, the inventors have found that this can be achieved by absorbing oxygen from an environment about a microbiological spore or a food product containing a spore.

The inventors have found that oxygen scavengers are very useful in embodiments of the invention. Oxygen scavengers are described further herein. Generally speaking, oxygen scavengers have effect in certain embodiments of the invention by absorbing, or otherwise extracting, withdrawing or depleting oxygen from an environment about a spore, or from the spore itself, so as to provide conditions in which the amount of oxygen available for consumption by the spore is limited.

It has been found that oxygen scavengers are very useful with the pressure treatments described herein in certain embodiments of the invention for inactivation of a microbiological spore by absorbing oxygen from an environment about a spore. In these embodiments, oxygen scavengers have effect in those environments that are essentially an atmosphere defined by a package, such as a package in which the product is to be sold, or those environments wherein the aggregation of things, conditions or influences surrounding a spore is the product itself.

Surprisingly, it has been found that elimination of oxygen is not essential to inactivate aerobic microbiological spores. What is more important is the maintenance of oxygen at a low level and in particular at a level at which the amount of oxygen available for consumption by the spore is limited. For example, an amount that is typically consumed by a spore in a germination process may be limited so as to prevent the germination process.

Further, it has been found that it is not essential that oxygen be depleted prior to high pressure treatment.

A number of advantages stem from the use of oxygen scavengers in the invention, including, for example, a capacity to maintain a minimal oxygen concentration across the shelf-life of a food product, an ability to control the timing of the oxygen absorption process independently of HPP by activating an oxygen scavenger at a selected time, and by selecting oxygen scavengers of particular scavenging rates, a capacity to reduce oxygen and to control the time point at which such reduction is to be achieved.

In certain embodiments there is provided a method for inactivating a microbiological spore including the steps of subjecting a microbiological spore to an ultra high pressure treatment and absorbing oxygen from an environment about the spore to at least limit the consumption of oxygen by the spore.

Typically an oxygen scavenger, as described further herein, is used to absorb oxygen from an environment about the spore.

Useful pressure treatment conditions are described further herein.

In one embodiment, the conditions provided are such that the quantity of oxygen absorbed or depleted from the environment prevents germination of all aerobic spores in the environment.

The spore may be located within or on the surface of a product, for example a food, pharmaceutical, cosmetic or medical product. Alternatively, it may be located in an environment about a product, for example, an environment defined by a package for the product.

Examples of food products include food ingredients, food additives such as flavours, sweeteners, colouring agents, preservatives and processed foods. More particularly, examples of food products are those that are formulated so that they do not support the growth of anaerobic spore forming bacteria. This includes all high acid foods such those with a pH of less than 4.6, but also any other food where parameters such as water activity, the presence of antimicrobials etc, prevent the growth of anaerobic spore forming bacteria.

Examples of pharmaceutical, cosmetic and medical products include tablets, creams, lotions, suppositories, potions, syrups, suspensions, powders and blood bags.

In certain embodiments there is provided a method for producing a packaged food product including the steps of adding a food product to a package and subjecting the food product to an ultra-high pressure treatment, wherein the food product is in an oxygen-scavenging environment either before or after the ultra-high pressure treatment, said ultra-high pressure treatment and said oxygen scavenging environment being selected for inactivation of a selected microbiological spore in the food product.

In one embodiment, the food product is subjected to the ultra-high pressure treatment prior to being added to the package. In an alternative embodiment, the food product is placed in the package and subsequently subjected to the ultra-high pressure treatment.

The food product may be placed in an oxygen scavenging environment by any suitable method. For example, the food product may be placed in close proximity to, or in contact with, an oxygen scavenging material. Alternatively, an oxygen scavenging compound, such as an enzyme or a chemical compound, may be mixed with the food product, or the food product may be placed in an oxygen scavenging package.

It is preferred that the food product is placed in packaging that includes an oxygen scavenging material.

The oxygen scavenging material is suitably an oxygen scavenging packaging material.

The oxygen scavenging may be initiated before the food is added to the package or it may be initiated after the food is added to the package. In embodiments where the food product and the package are subjected to the ultra-high pressure treatment, the oxygen scavenging may be initiated before the ultra-high pressure treatment or after the ultra-high pressure treatment.

In an especially preferred embodiment, the food product is placed in a package that includes an oxygen scavenging material, and the food and package are subjected to the ultra-high pressure treatment.

Most preferably, the oxygen-scavenging environment is maintained after the ultra high pressure treatment has been completed.

It is preferred that the packaging provides a barrier to oxygen permeability. Suitably, the packaging may be monolayer or multilayer, and includes at least one layer with an oxygen scavenging ability and at least one layer that provides a barrier to oxygen entering the packaging from the external outside the packaging. It will be understood that when packaging is a monolayer, the monolayer may have both oxygen scavenging and oxygen barrier capacities.

Also provided in certain embodiments is, in a process for manufacture of a food product, the steps of subjecting a food to an ultra-high pressure treatment and absorbing oxygen from an environment about the food product to provide conditions for limiting the consumption of oxygen by a microbiological spore in the environment.

In one embodiment, the ultra-high pressure treatment is applied before oxygen is absorbed from an environment about the food product.

Typically an oxygen scavenger, as described further herein, is used to absorb oxygen from an environment about the food product.

Where an oxygen scavenger is used to absorb oxygen from an environment about the food product, oxygen may be absorbed at a selected time, either before or after the pressure treatment by activation of the oxygen scavenger.

Useful pressure treatment conditions are described further herein.

In one embodiment, the environment about the food product is defined by a package in which the food product is to be sold.

In one embodiment, the process includes the further step of providing conditions for limiting germination or growth of an anaerobic microbiological spore in the environment.

In another embodiment, the process includes a further step of contacting the food with a compound for inactivating a spore, such as for example an enzyme, such as chitinase. Other compounds include bacteriocins.

In certain embodiments there is provided a food product manufactured by the above described process.

The food product may be provided in a package in which it is to be sold.

In certain embodiments there is provided a use of an oxygen scavenger for inactivating a microbiological spore in an ultra-high pressure treatment of a food product.

The oxygen scavenger is used to inactivate a microbiological spore by absorbing oxygen from an environment about the food product to provide conditions for limiting the consumption of oxygen by a microbiological spore in the environment.

Preferably, the oxygen scavenger is used in an amount or otherwise applied such that the quantity of oxygen absorbed or depleted from the environment about the food product prevents germination of all aerobic spores in the environment.

The oxygen scavenger may be included in the food product, for example as an ingredient for the manufacture of the food product. Alternatively, it may be located in a sachet adjacent to the food product. Still further, the oxygen scavenger may be included in a package for the food product, including for example a package in which the food product is to be sold.

Examples of suitable oxygen scavengers are described further herein.

Also provided is an ultra-high pressure treatment adapted for inactivating a microbiological spore in a food product, the treatment including the step of absorbing oxygen from an environment about the food product to provide conditions for limiting the consumption of oxygen by a microbiological spore in the environment.

Typically an oxygen scavenger, as described further herein, is used to absorb oxygen from an environment about the food product.

In one embodiment, the ultra-high pressure treatment is applied before oxygen is absorbed from an environment about the food product.

Typically an oxygen scavenger, as described further herein, is used to absorb oxygen from an atmosphere about the food product.

Where an oxygen scavenger is used to absorb oxygen from an environment about the food product, oxygen may be absorbed at a selected time, either before or after the pressure treatment by activation of the oxygen scavenger.

Useful pressure treatment conditions are described further herein.

In one embodiment, the environment about the food product is defined by a package in which the food product is to be sold.

Also provided is a method for achieving commercial sterility of a product (i.e. preventing microbiological growth in or on the product) including the steps of subjecting a product to an ultra-high pressure treatment and absorbing oxygen from an environment about the product to provide conditions for limiting the consumption of oxygen by a microbiological spore in the environment.

Typically an oxygen scavenger, as described further herein, is used to absorb oxygen from an atmosphere about the product.

Useful pressure treatment conditions are described further herein.

In one embodiment, the conditions provided are such that the quantity of oxygen absorbed or depleted from the environment prevents germination of all aerobic spores in the environment.

The spore may reside within or on the surface of a product, for example a food, pharmaceutical, cosmetic or medical product. Alternatively, it may be located in an atmosphere about a product, for example, an atmosphere defined by a package for the product.

Examples of food products include food ingredients, food additives such as flavours, sweeteners, colouring agents, preservatives and processed foods.

Examples of pharmaceutical and cosmetic products include tablets, creams, lotions, suppositories, potions, syrups, injectable compositions such as vaccines and intravenous infusions.

An oxygen scavenger is typically a molecule that is capable of absorbing oxygen to provide an environment having an oxygen concentration that is lower than would be the case but for the oxygen scavenger. Suitable oxygen scavengers can, for example, include, without limitation: (i) an oxidisable compound and a transition metal catalyst, (ii) an ethylenically unsaturated hydrocarbon and a transition metal catalyst, (iii) an ascorbate, (iv) an isoascorbate, (v) a sulfite, (vi) an ascorbate and a transition metal catalyst, (vii) a reducible organic compound such as a quinone, a photoreducible dye, or a carbonyl compound, (viii) a tannin, (ix) biological systems such as enzymes and (x) rusting of finely divided iron particles.

According to the invention, oxygen scavenging may be provided by oxidisable solids, for example porous sachets containing iron powder. In another embodiment, oxidisable MXD-6 Nylon may blended with polyester in the walls of flow-moulded containers. The effectiveness of this depends on the presence of a cobalt salt catalyst. Further embodiments include sandwiching crystalline oxidisable material between the layers of multilayer containers, and including a catalyst for the reaction of oxygen with hydrogen in a sandwich arrangement as above or as a deposit on the inner surface of the package.

In further embodiments, oxygen scavenging may be implemented as disclosed in Rooney, M. L., Chemistry and Industry, 20 Mar. 1982, pp. 197-198. These embodiments involve the inclusion of a photo-oxidizable rubber and a photosensitising dye into a polymer film packaging material and then exposing it to visible light.

In further embodiments, oxygen scavenging may be implemented as disclosed in Rooney, M. L. and Holland, R. V., Chemistry and Industry, 15 Dec. 1979, pp. 900-901 and Rooney, M. L., Journal of Food Science, Vol. 47, No. 1, pp. 291-294, 298. These embodiments initiate oxygen scavenging upon illumination and require constant illumination of the package in order to maintain the scavenging effect.

In still further embodiments, oxygen scavenging may be implemented as disclosed in U.S. Pat. No. 5,211,875, the entire contents of which are incorporated by cross-reference. These embodiments avoid the problem of oxygen-sensitivity prior to use, involving an oxidisable organic compound (typically 1,2-Polybutadiene) and a transition metal catalyst (typically cobalt salt). Oxygen scavenging is initiated by exposing the composition to an electron beam, or ultraviolet or visible light.

U.S. Pat. Nos. 5,958,254, 6,346,200, 6,517,728, 6,746,630, 6,123,901 and 6601732 the entire contents of which are herein incorporated by cross-reference, describe a solid phase composition for reducing the concentration of ground state molecular oxygen present in an environment or liquid comprising at least one reducible organic compound which is reduced when the composition is subjected to predetermined conditions, the reduced form being oxidisable by ground state molecular oxygen, wherein the reduction and/or subsequent oxidation of the organic compound occurs independent of both constant illumination with visible light and the presence of a transition metal catalyst.

The solid phase composition of U.S. Pat. Nos. 5,958,254, 6,346,200, 6,517,728, 6,746,630, 6,123,901 and 6,601,732 allow the oxygen scavenging ability to be activated when desired by the user by exposing the composition to the predetermined conditions to reduce the organic compound to an oxidisable exposure to light of a certain intensity or wavelength, by the application of heat, γ-irradiation, corona discharge or an electron beam. The organic compound may be reduced by incorporating in the composition a reducing agent which in turn can be activated under predetermined conditions, say, by heating.

The composition described in U.S. Pat. Nos. 5,958,254, 6,346,200, 6,517,728, 6,746,630, 6,123,901 and 6,601,732 may be provided in the form of a packaging film or laminate.

Pressure treatments in accordance with certain embodiments of the invention are in excess of 100 MPa (14,500 psi), preferably in the range of from 300-1000 MPa (43,500-145,000 psi). Particularly preferred are pressure treatments in the range of from 400-800 MPa (58,000-116,000 psi), even more preferably, from 500-700 MPa (72,500-101,500 psi). These treatments may be applied in accordance with standard processes, for example as in U.S. Pat. Nos. 6,207,215 and 6,086,936, the entire contents of which are herein incorporated by cross-reference.

The temperature used in this process is not especially critical. The initial temperature at the start of high pressure treatment may range from 0 to 75° C., more preferably from ambient temperature to 60° C.

In certain embodiments a variety of foods can be treated including without limitation vegetables, fruits, nuts, meats and fish, dairy, eggs, food products such as processed foods containing these as ingredients including processed meals, sauces, soups, stews, beverages and juices, and various food additives including flavours, colours, preservatives and the like.

Both high acid foods (i.e. having a pH less than 4.6) and low acid foods (i.e. having a pH greater than or equal to 4.6) can be treated in certain embodiments.

The invention is described below by reference to certain non-limiting examples. It will be appreciated by persons skilled in the art that numerous variations and/or modifications may be made to the invention as described in the examples without departing from the spirit or scope of the invention as broadly described. The following examples are, therefore, to be considered in all respects as illustrative and not restrictive.

### EXAMPLES

### Example 1

This example demonstrates the high pressure treatment of heat-resistant moulds in three different packaging films. High pressure (HP) treatment was carried out in a 2 L high pressure processing (HPP) unit.

Material and Methods

Cultures: Two heat-resistant mould species, Byssochlamys fulva FRR 3792 from heat processed strawberry purée, and Neosartorya fischeri FRR 4595 from heated processed strawberry purée, were used.

Packaging films: Three packaging films were used to prepare pouches used for this example: (i) OPET//EVOH//O₂-scavenger//CPP multilayer laminate, hereafter referred to as “OS laminate”, in which the oxygen-scavenger material is based on a reducible organic compound such as a quinone, a photoreducible dye, or a carbonyl compound; (ii) EVA monolayer film, which is highly permeable to O₂; (iii) a heat-sealable laminate comprising a layer of EVOH, hereafter referred to as “EVOH laminate” which has a low permeability to O₂ and is commonly used as an oxygen barrier film. (OPET=oriented polyester, EVOH=ethylene vinyl alcohol, CPP=cast polypropylene, EVA=ethylene vinyl acetate).

Procedure

Fungi were grown on MEA at 30° C. for 3 weeks and spore suspensions (10⁴ cfu/mL) were prepared in 20° Brix syrup with citric acid, pH 4.2. Spore suspensions of B. fulva and N. fischeri were blanched at 95° C. for 5 minutes. 5 mL of blanched sample was poured into each of 15 pouches (5 cm×10 cm) made from the 3 types of film (i.e. OS laminate in which the oxygen scavenging process was triggered prior to HP treatment, EVA and EVOH laminate) for each fungal species. These pouches were then BP treated at 600 MPa for 0 (control), 1 and 2 minutes at ambient temperature (approx. 25° C.), giving 5 pouches of each packaging film type per treatment for each mould species. After HPP, the pouches were incubated for 2 weeks at 30° C., then assessed for mould growth.

Results

After 2 weeks' incubation at 30° C., growth consistent with >10⁶ cfu/mL was observed in all EVA and EVOH laminate pouches that had not been subjected to HP treatment (control pouches). No visible growth was observed in the OS laminate control pouches.

Similarly, for pouches that had received the HP treatment of 600 MPa for 1 and 2 minutes, growth consistent with >10⁶ cfu/mL was observed in all EVA and EVOH laminate pouches for both fingi at both pressure treatment times, whereas no visible growth was apparent in any of the OS laminate pouches.

Inoculum Viability Test

After 2 weeks' incubation, pouches containing N. fischeri and B. fulva in OS laminate packages were opened and 0.1 mL samples were plated out onto duplicate plates of DRBC and MEA to check the inoculum viability. All plates were incubated at 30° C. for 5 days. The results (averages of the duplicate plates) are as follows:

TABLE 1
Survival of heat-resistant mould spores
in OS laminate pouches after HPP

| HPP Treatment | B. fulva | N. fischeri |
|---|---|---|
| (minutes) | (cfu/mL)* | (cfu/mL)* |
| 0 | 260 | 840 |
| 1 | 53 | 50 |
| 2 | 10 | 200 |

*average of 2 duplicate plates

Discussion

A significant reduction in numbers of both B. fulva and N. fischeri in the pouches prepared using the OS laminate was observed for all pressure treatments (including the control) compared with the other two types of pouches. The consistently low numbers recovered from the OS laminate pouches after 2 weeks' incubation indicate that, although some ascospores survive the pressure treatment, they are prevented from growing in the OS laminate pouches. Furthermore, the combined use of oxygen scavenging and HPP was found to provide enhanced reduction in growth of both spore types compared with use of oxygen scavenger alone. In contrast, visible growth consistent with >10⁶ cfu/mL was observed after 2 weeks' incubation at 30° C. for both fungi in the other two types of film (EVA, EVOH laminate) after both pressure treatments.

### Example 2

This example demonstrates the combined effect over time of high pressure treatment and oxygen depletion using an oxygen scavenger on the survival of one heat-resistant mould and one heat-resistant Bacillus species. High pressure processing was carried out in a 2 L high pressure processing (HPP) unit.

Material and Methods

Cultures: A heat-resistant mould species, Neosartorya fischeri FRR 4595 from heated strawberry purée, and a heat-resistant bacterial species, Bacillus subtilis FRR B2738 from marinade were used.

Packaging films: Two packaging films with low oxygen permeabilities were used for this evaluation, namely the OS laminate and the EVOH laminate referred to in Example 1.

Procedure

The mould species N. fischeri was grown on MEA at 30° C. for 11 weeks to obtain ascospores with high pressure resistance. A spore suspension (approximately 3×10³ cfu/mL) was prepared in Tryptone Glucose Yeast extract (TGY) broth, pH 4.5. A spore suspension of B. subtilis (2×10² cfu/mL) was similarly prepared in Nutrient broth, pH 4.5. For each test organism, 5 mL of spore suspension was poured into pouches (5 cm×10 cm) prepared using each film (OS laminate and EVOH laminate). In this experiment, the oxygen scavenging process in the pouches prepared using the OS laminate was activated prior to high pressure treatment. Multiple pouches were prepared for each species to enable duplicate sampling throughout the post-treatment incubation period. These pouches were then HP treated at 600 MPa for 3 minutes at ambient temperature (approx. 25° C.). After HPP, the treated pouches were incubated at 30° C. Spores in control pouches, which were not subjected to HP treatment, were also monitored for growth.

Viable counts were determined immediately prior to high pressure treatment, within 24 hours after high pressure treatment (storage at 2° C.), then at two-weekly intervals thereafter. Pouches were examined for visible growth, then viable counts determined by dilution plating of the pouch contents onto suitable growth media, and incubation of the plates at 30° C.

Results

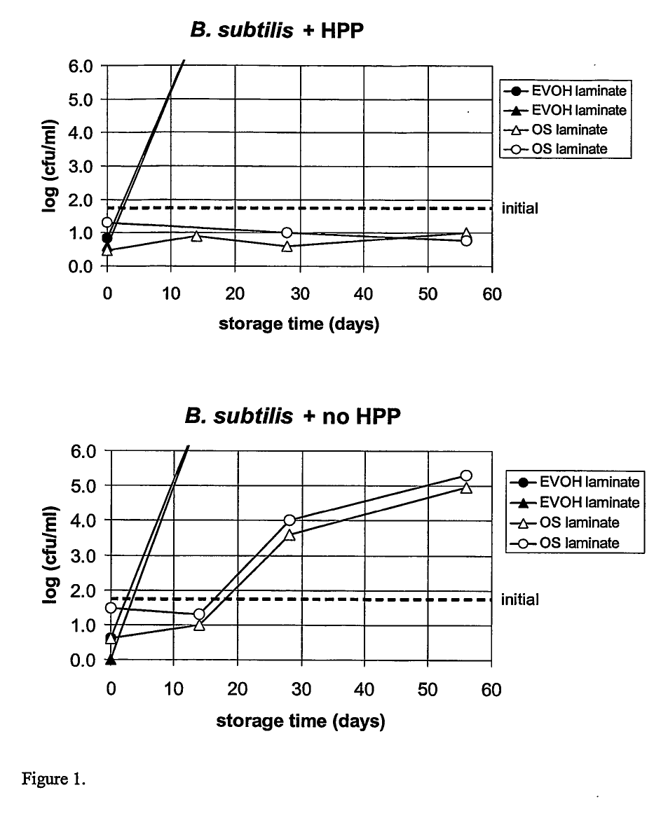

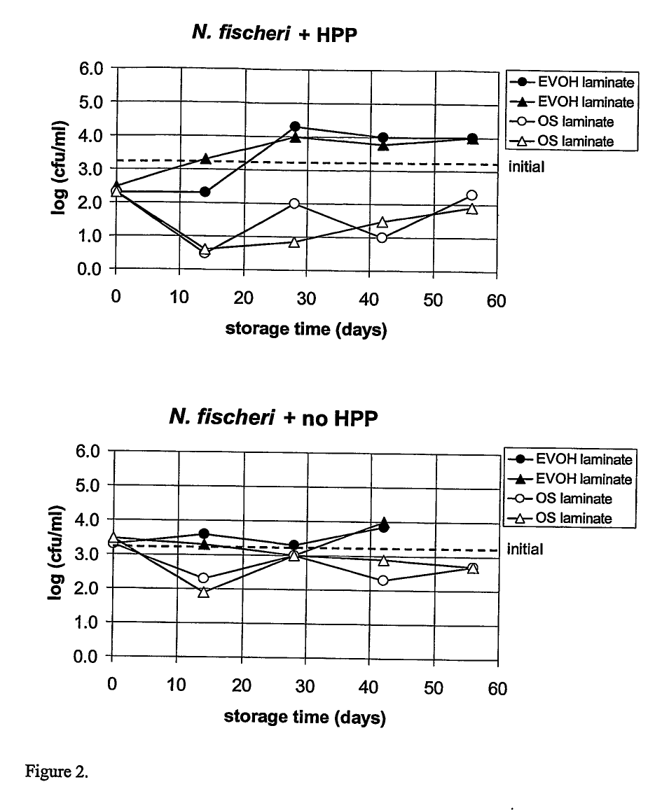

The results for the duplicate sets of pouches containing suspensions of B. subtilis and N. fischeri subjected to HPP, together with control pouches (no HPP), are shown in FIGS. 1 and 2, respectively, and the data is summarised in Table 2. After 2 weeks' incubation at 30° C., growth consistent with >10⁶ cfu/mL was observed for B. subtilis in the EVOH laminate pouches. The EVOH laminate pouches inoculated with this Bacillus species were discarded after 2 weeks' incubation because of the high numbers of viable cells. In the OS laminate pouches, numbers of B. subtilis were found to be significantly reduced in both HP treated and control pouches.

After 2 weeks' incubation at 30° C., the mould N. fischeri did not appear to have grown in the EVOH laminate pouches, and its numbers were distinctly lower in the OS laminate pouches.

After 4 weeks' incubation, the number of viable spores of B. subtilis in the HP treated OS laminate pouches was significantly lower than in the control OS laminate pouches. Numbers of viable ascospores of the mould N. fischeri in the HP treated OS laminate pouches were also substantially lower than in the other pouches.

Discussion

As seen in FIGS. 1 and 2, the growth of spores of B. subtilis and the heat-resistant mould N. fischeri in the EVOH laminate pouches was essentially independent of HP treatment. While some growth inhibition was observed for the spores in the control OS laminate pouches (i.e. not subjected to HPP), a significant reduction in numbers of both N. fischeri and B. subtilis was observed for the spores contained in the pouches comprising the OS packaging film that were subjected to HPP. These results demonstrate clearly the significant advantage of using the combination of oxygen scavenger and HPP to reduce the growth of the ascospores of N. fischeri and the spores of B. subtilis compared with using either treatment alone.

TABLE 2
Growth of microbiological spores after
HPP with and without oxygen scavenger

| | Incubation time | EVOH laminate | OS laminate |
|---|---|---|---|
| Species | (weeks) | (cfu/mL) * | (cfu/mL) * |
| N. fischeri | 0 | 250 | 200 |
| | 2 | 1,100 | 4 |
| | 4 | 15,000 | 54 |
| | 6 | 8,000 | 20 |
| | 8 | 10,000 | 140 |
| B. subtilis | 0 | 6 | 12 |
| | 2 | >1,000,000 | 8 |
| | 4 | Not tested | 6 |
| | 8 | Not tested | 8 |

* average of two replicates

### Example 3

This example demonstrates the effect of the sequence of triggering the oxygen scavenging activity of the OS laminate pouches in relation to the high pressure treatment. The oxygen scavenging activity of the pouches was triggered either before or after the high pressure treatment. High pressure treatment was carried out in 2 L high pressure processing (HPP) unit.

Materials and Methods

Cultures: As with the above example, one heat-resistant mould species, Neosartorya fischeri FRR 4595 from heated strawberry purée, and one heat-resistant bacterial species, Bacillus subtilis FRR B2738 from marinade, were used.

Packaging material: The packaging film used to prepare the pouches was the OS laminate referred to above.

Procedure

The mould species was grown on MEA at 30° C. for 11 weeks to obtain ascospores with high pressure resistance. A spore suspension (approximately 3×10³ cfu/mL) was prepared in Tryptone Glucose Yeast extract (TGY) broth, pH 4.5. A spore suspension of B. subtilis (2×10² cfu/mL) was similarly prepared in Nutrient broth, pH 4.5. For each test organism, 5 mL of spore suspension was poured into pouches (5 cm×10 cm) prepared using OS laminate. Multiple pouches were prepared for each species to enable duplicate sampling throughout the post-treatment incubation period.

The oxygen scavenging activity of one set of the OS laminate pouches was activated prior to filling with the test organisms for the high pressure treatment. In the second set of pouches the oxygen scavenging activity of the OS laminate pouches was activated immediately after high pressure treatment.

The pouches were BP treated at 600 MPa for 3 minutes at ambient temperature (approx. 25° C.). After HPP, the pouches were incubated at 30° C. Viable counts were determined immediately prior to high pressure treatment, within 24 hours after high pressure treatment (storage at 2° C.), then at two-weekly intervals thereafter. Pouches were examined for visible growth, then viable counts determined by dilution plating of the pouch contents onto suitable growth media, and incubation of the plates at 30° C.

Results

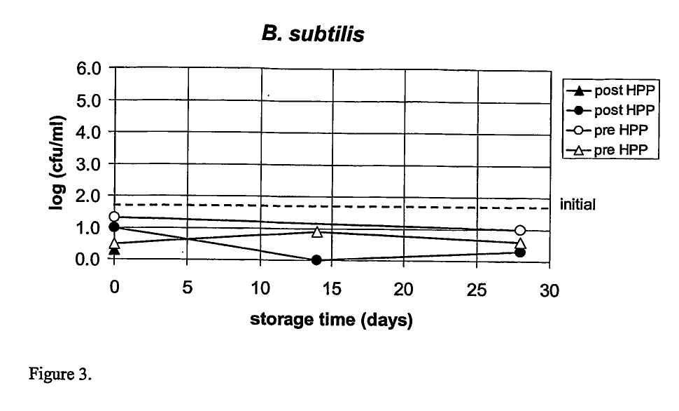

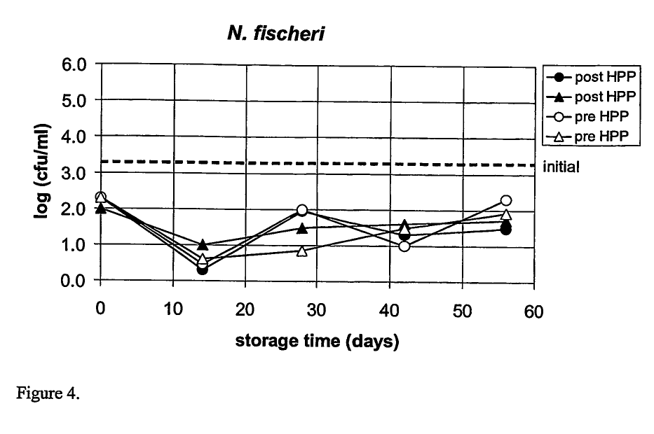

The results for the duplicate sets of OS laminate pouches containing suspensions of B. subtilis and N. fischeri, which were triggered before or after HPP, are shown in FIGS. 3 and 4, respectively, and the data is summarised in Table 3.

TABLE 3
Effect of sequence of triggering oxygen scavenging activity
and HPP on viability of microbiological spores

| | Incubation time | triggered pre-HPP | triggered post-HPP |
|---|---|---|---|
| Species | (weeks) | (cfu/mL) * | (cfu/mL) * |
| N. fischeri | 0 | 200 | 150 |
| | 2 | 4 | 6 |
| | 4 | 54 | 60 |
| | 6 | 20 | 30 |
| | 8 | 140 | 40 |
| B. subtilis | 0 | 12 | 6 |
| | 2 | 8 | <1 |
| | 4 | 6 | 2 |

* average of two replicates

Discussion

The level of reduction in the numbers of both types of spores was found to be practically independent of whether the oxygen scavenging process in the OS laminate pouches commenced prior to HPP or after HPP. These results indicate that the enhanced reduction of spore growth obtained using the combination of oxygen scavenging and HPP can be achieved independent of the sequence in which these two steps are performed.

Those skilled in the art will appreciate that the present invention may be susceptible to variations and modifications other than those specifically described. It is to be understood that the present invention encompasses all such variations and modifications that fall within its spirit and scope.

1. A method for inactivating a microbiological spore including the steps of subjecting a microbiological spore to an ultra high pressure treatment and absorbing oxygen from an environment about the spore to at least limit the consumption of oxygen by the spore.

2. A method according to claim 1 wherein oxygen is absorbed from an environment about the spore by an oxygen scavenger.

3. A method according to claim 2 wherein the oxygen scavenger is selected from the group consisting of (i) an oxidisable compound and a transition metal catalyst, (ii) an ethylenically unsaturated hydrocarbon and a transition metal catalyst, (iii) an ascorbate, (iv) an isoascorbate, (v) a sulfite, (vi) an ascorbate and a transition metal catalyst, (vii) a reducible organic compound such as a quinone, a photoreducible dye, or a carbonyl compound, (viii) a tannin, (ix) biological systems such as enzymes and (x) rusting of finely divided iron particles.

4. A method according to claim 2 wherein oxygen is absorbed before the ultra high pressure treatment.

5. A method according to 1 wherein the spore is within or on the surface of a product.

6. A method according to claim 5 wherein the product has a pH less than 4.6.

7. A method according to claim 6 wherein the environment about the spore is an atmosphere defined by a package for the product.

8. A method according to claim 7 wherein the package has an oxygen scavenger.

9. A method according to claim 1 wherein the microbiological spore is partially germinated.

10. A method according to claim 1 wherein the ultra high pressure treatment is in the range of 300 MPa to 1000 MPa.

11. A method according to claim 5 wherein the product is heated before the ultra high pressure treatment.

12. A method for producing a packaged food product including the steps of adding a food product to a package and subjecting the food product to an ultra-high pressure treatment, wherein the food product is in an oxygen-scavenging environment either before or after the ultra-high pressure treatment, said ultra-high pressure treatment and said oxygen scavenging environment being selected for inactivation of a microbiological spore in the food product.

13. In a process for manufacture of a food product, the steps of subjecting a food to an ultra-high pressure treatment and absorbing oxygen from an environment about the food to provide conditions for limiting the consumption of oxygen by a microbiological spore in the environment.

14. A food product manufactured by the process of claim 13.

15. A use of an oxygen scavenger for inactivating a microbiological spore in an ultra-high pressure treatment of a food product.

16. An ultra-high pressure treatment adapted for inactivating a microbiological spore in a food product, the treatment including the step of absorbing oxygen from an environment about the food product to provide conditions for limiting the consumption of oxygen by a microbiological spore in the environment.

17. A method for achieving commercial sterility of a product including the steps of subjecting a product to an ultra-high pressure treatment and absorbing oxygen from an environment about the product to provide conditions for limiting the consumption of oxygen by a microbiological spore in the environment.

18. A product produced by the method of claim 17.
